(19) United States
(12) Patent Application Publication
(10) Pub. No.: US 2022/0142698 A1
(43) Pub. Date: May 12, 2022

(54) ELECTROSURGICAL INSTRUMENT

(71) Applicant: Erbe Elektromedizin GmbH, Tuebingen (DE)

(72) Inventor: Felix Bob, Rottenburg (DE)

(73) Assignee: Erbe Elektromedizin GmbH, Tuebingen (DE)

(21) Appl. No.: 17/520,260

(22) Filed: Nov. 5, 2021

(30) Foreign Application Priority Data
Nov. 12, 2020 (EP) 20207244.3

(51) Int. Cl. A61B 18/08

(52) U.S. Cl. CPC A61B 18/085; A61B 2018/00601

(57) ABSTRACT

A surgical instrument having a first jaw and a second jaw arranged in a hinged manner with respect to each other. A tool is slidably supported in a longitudinal direction in a tool channel, preferably in or on the first jaw. The tool may be moved in longitudinal direction by an actuation device. The actuation device has an actuation element and a drive element that are pivotably supported around a pivot axis, preferably on the first jaw. A rotational coupling may be established between the actuation element and the drive element via a coupling device.

### CROSS-REFERENCE TO RELATED APPLICATION

This application claims priority to European Application No. 20207244.3, filed Nov. 12, 2020, the entirety of which is incorporated by reference herein.

### TECHNICAL FIELD

Embodiments of the invention relate to an electrosurgical instrument having two jaws that are pivotably supported on one another by means of a hinge.

### BACKGROUND

EP 2 436 327 A1 describes a surgical instrument having two jaws and a movable supported knife. In some embodiments a blocking element is provided that blocks the movement of an actuation element in the open position of the jaws, wherein the actuation element can be pivoted for movement of the knife. In a release position of the blocking element pivoting of the actuation element is allowed such that the knife can be extended in longitudinal direction. In another embodiment pivoting of actuation element is hindered, if the jaws are opened in that a resetting element is biased in the open position of the jaws such that a remarkably increased force would be necessary to pivot the actuation element. In addition, an embodiment is provided having a linearly movable actuation element in which the movement coupling with the knife is established only if the jaws are closed.

US 2017/0128120 A1 describes a surgical instrument having two jaws and a linearly movable actuation element for moving a knife. The actuation element comprises a first toothed rack in engagement with a gear. The actuation element and the gear are arranged on the second jaw. The knife and a second toothed rack immovably coupled with the knife are arranged on the first jaw. The gear and the second toothed rack are disengaged in the open position and only engaged in the closed position, such that only in the closed position a movement of the knife is effected, if the actuation element is moved.

US 2016/0302844 A1 discloses a surgical instrument having two jaws. A linearly movable actuation element for movement of a knife is coupled with the knife by means of a coupling device, if the jaws are in the closed position. This principle corresponds to an embodiment of EP 2 436 327 A1 having a linearly movable actuation element.

Frequently a linearly movable actuation element is undesired for ergonomic reasons. In addition, relatively much space is required on the instrument in order to provide the linear moving path for the actuation element. Thereby the shank sections of the jaws frequently get very long. In turn, this has effects on the opening angle of the jaw sections of the jaws, if the shank sections are moved away from one another at the proximal end.

### SUMMARY

Thus, it can be considered an object of embodiments of the present invention to provide a surgical instrument having a pivotable actuation element that guarantees high operational safety with simple configuration.

This object may be solved by means of a surgical instrument having the features of claim 1.

The surgical instrument, according to an embodiment of the invention, comprises a first jaw and a second jaw that are supported on one another in a hinged manner. Each jaw has a jaw section having a tissue contact surface in the distal end section. Each jaw section is connected with a shank section of the respective jaw. In the area of the hinge of the jaws the shank section and the jaw section transition into one another or are connected with one another.

The tool is movably supported on the first jaw in a guided manner in longitudinal direction. In addition, an actuation element and a drive element are pivotably supported around a pivot axis on the first jaw. The drive element is movably coupled with the tool, e.g. by means of a transmission device such as a gearing mechanism, particularly lever gearing mechanism.

The instrument also comprises a coupling device having a coupling element. The coupling element is movably supported between a coupling position and a decoupling position. In the coupling position the coupling element establishes a rotational coupling or a torque-proof connection between the actuation element and the drive element. Thus, if a pivot movement of the actuation element around the pivot axis occurs, also the drive element pivots around the pivot axis. In the decoupling position this rotational coupling or torque-proof connection is suspended and allows a relative rotation between the actuation element and the drive element. If the actuation element is pivoted around the pivot axis in the decoupling position, the drive element is not influenced thereby. The drive element can remain in its initial position independent from pivot movement of actuation element, if the coupling element is in the decoupling position.

The coupling element is pivotably supported around the pivot axis together with the actuation element. The coupling element is movably arranged on the actuation element. Particularly establishing the rotational coupling or the movement of the coupling element in the coupling position is only possible, if the actuation element as well as the drive element are in their respective initial position.

In the coupling position a rotational coupling is preferably established in both rotational directions or in both senses of rotation around the pivot axis.

The coupling element can be manually moved between the coupling position and the decoupling position, e.g. by indirect actuation by means of a finger. As an alternative or in addition, the coupling element can be actuated or moved by means of the second jaw. For this purpose the surgical instrument is particularly configured to move the coupling element from the decoupling position into the coupling position, if the jaws are pivoted relative to one another in the closed position. During this movement in the closed position the second jaw gets directly or indirectly in contact with the coupling element and moves the coupling element into the coupling position.

The coupling element is preferably moved from the coupling position into the decoupling position, if the jaws are moved from the closed position into the open position and if the actuation element and the drive element are in their respective initial position. If the coupling element establishes a torque-proof coupling between the actuation element and the drive element in the coupling position, the coupling element is preferably held in the coupling position by force-fit and/or form-fit, even though the jaws are moved from the closed position into the open position.

It is ensured in all embodiments that the tool, e.g. a knife, is not actuated unintentionally. In the decoupling position the actuation element can be pivoted without tool movement occurring in the longitudinal direction. If the coupling element is, however, moved in the coupling position, the actuation of the actuation element results in a pivot movement of the drive element around the pivot axis that is in turn movably coupled with the tool and initiates in this manner a tool movement.

In a preferred embodiment the drive element comprises a coupling recess. The coupling element engages the coupling recess in the coupling position. In doing so, a torque-proof connection between the drive element and the actuation element is created. In the decoupling position the coupling element is located completely outside the coupling recess. The coupling recess is preferably limited by flanks opposite to one another in circumferential direction around the pivot axis. A coupling device configured in this manner can be realized with a few components in a simple manner.

In a preferred embodiment of the surgical instrument the coupling element can be slidable or linearly movable between the coupling position and the decoupling position. In an alternative embodiment the coupling element can be pivotable between the coupling position and the decoupling position. As an alternative to this, also superimposed linear and pivot movements can be realized. Independent from the movement path of the coupling element, it extends at least in its end section prior to reaching the coupling position substantially orthogonal or radial to the pivot axis.

It is advantageous, if a biasing device is provided. The biasing device is configured to create a biasing force on the coupling element. By means of the biasing force the coupling element is urged into the decoupling position. The biasing force can be a pushing force and/or traction force. For example, the biasing device can comprise a spring element for creation of the biasing force. One single spring element is sufficient.

In an embodiment the spring element and the coupling element can be directly connected with one another. The connection can be configured as releasable connection or non-releasable connection. It is, for example, possible to connect the coupling element and the spring element with each other by means of a substance bond connection and/or an adhesive connection. For example, the spring element can be connected with the coupling element by means of an adhesive, by overmolding or overpouring.

In addition or as an alternative, the spring element can be releasably or non-releasably arranged on the actuation element, e.g. by means of the substance bond connection and/or an adhesive connection. For example, the spring element can be connected with the actuation element by means of an adhesive, by overmolding or overpouring.

In a preferred embodiment the spring element and the coupling element can be configured integrally. The spring element and the coupling element can transition into one another without seam or joint. In this configuration it is advantageous, if the spring element and the coupling element consist, for example, of metal or metal alloy. They can be manufactured by separation and/or deformation from a sheet metal part or a wire.

It is preferred, if the spring element is a flexible spring or leaf spring. The flexible spring or leaf spring can create a biasing force orthogonal or radial to the pivot axis. The flexible spring can extend, for example, in an arc-shaped manner around the pivot axis, particularly in an angle range of at least 90° or at least 120° or at least 180°. The leaf spring can extend straight or curved from an attachment end up to a free end. The attachment end of the leaf spring can have a longer distance to the pivot axis than the free end.

As an alternative to this the spring element can be a helical spring. A helical spring can be orientated orthogonal or radial to the pivot axis, for example. In all embodiments of the spring element the spring element is supported on one side on the actuation element and on the other side on the coupling element.

In a preferred embodiment the surgical instrument comprises a resetting device. The resetting device is configured to urge the actuation element and/or the drive element in an initial position. For this, the resetting device can have a first resetting element for the drive element and/or a second resetting element for the actuation element. It can be advantageous, if the first resetting element is formed by an elastically deformable transmission element that is arranged between the drive element and the tool, e.g. an elastically deformable connecting rod.

If only one single resetting element for the actuation element is provided, the resetting force is not applied on the drive element in the decoupling position of the coupling element. In order to avoid an unintentional movement of the tool, the drive element can be secured against a pivot movement around the pivot axis in the decoupling position of the coupling element. For this purpose the coupling element can be used in the decoupling position, for example. Preferably the coupling element engages the jaw and the drive element in the decoupling position, however, does not engage the actuation element.

Embodiments described herein include surgical instruments having a first jaw and a second jaw that may be moved between an open position and a closed position. Each jaw may include a jaw section and a shank section that is immovably connected with the jaw section, wherein the two shank sections of the two jaws serve for their actuation. For example, the surgical instrument can be configured in a forceps-like or scissors-like manner.

In addition, embodiments of the surgical instrument may have a tool, e.g. a knife, that is movably supported in or on the first jaw in a guided manner in longitudinal direction. The first jaw may have a tool channel or a knife channel for this purpose.

In such surgical instruments it is desirable to avoid an undesired tool actuation. It is particularly desired to hinder or block the extension of the tool from the tool channel, if the jaws are opened.

### BRIEF DESCRIPTION OF THE DRAWINGS

Embodiments of the invention are derived from the dependent claims, the description and the drawings. In the following, embodiments of the invention are explained in detail based on the attached drawings. The drawings show:

FIGS. 1-3 a schematic side view of a surgical instrument, particularly an electrosurgical instrument having a movable tool and an actuation device for movement of the tool in different conditions of the actuation device,

FIG. 4 an embodiment of an actuation device for the electrosurgical instrument of FIGS. 1-3 in a schematic side view, wherein a coupling device of the actuation device is in a decoupling condition,

FIG. 5 the actuation device of FIG. 4, wherein the coupling device is in a coupling condition,

FIG. 6 an embodiment of an actuation device for the electrosurgical instrument of FIGS. 1-3 in a schematic side view, wherein the coupling device comprises a movable coupling element that is in a decoupling position,

FIG. 7 the actuation device of FIG. 6, wherein the coupling element is in a coupling position,

FIG. 8 an embodiment of an actuation device for the electrosurgical instrument of FIGS. 1-3 in a schematic side view, wherein a coupling device comprises a movable coupling element that is in a decoupling position,

FIG. 9 the actuation device of FIG. 8, wherein the coupling element is in a coupling position,

FIG. 10 an embodiment of an actuation device for the electrosurgical instrument of FIGS. 1-3 in a schematic side view, wherein the coupling device comprises a movable coupling element that is in a decoupling position,

FIG. 11 the actuation device of FIG. 10, wherein the coupling element is in a coupling position,

FIG. 12 an embodiment of an actuation device for the electrosurgical instrument of FIGS. 1-3 in a schematic side view, wherein the coupling device comprises a movable coupling element that is in a decoupling position,

FIG. 13 the actuation device of FIG. 12, wherein the coupling element is in a coupling position,

FIG. 14 the actuation device of FIGS. 12 and 13, wherein the coupling element is in a decoupling position and the actuation element of the actuation device is pivoted out of an initial position,

FIG. 15 a further embodiment of the actuation device based on the embodiments of FIGS. 8 and 9, wherein the coupling element is in a decoupling position and the actuation element of the actuation device is in the initial position.

### DETAILED DESCRIPTION

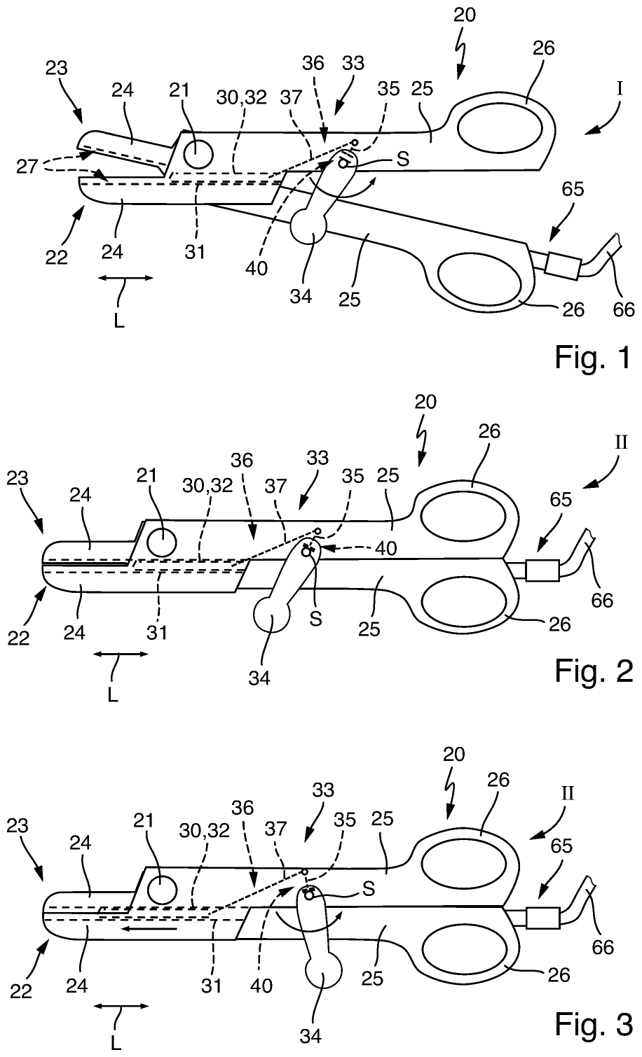

FIGS. 1-3 schematically illustrate an embodiment of a surgical instrument 20. The surgical instrument 20 is particularly configured as reusable instrument. Alternatively to this, the surgical instrument could also be realized as single-use instrument.

As illustrated, the surgical instrument 20 is configured in the type of scissors or forceps in the embodiment. It comprises a hinge 21 by means of which a first jaw 22 and a second jaw 23 are pivotably supported on one another. Each jaw 22, 23 comprises a jaw section 24 in the distal area. In addition each jaw 22, 23 comprises a shank section 25, wherein the two shank sections 25 are configured for actuation of the jaw sections 24. For this each shank section 25 has a handle piece 26 at the proximal end. The handle pieces 26 of the two jaws 22, 23 can be moved away or toward one another, whereby the jaw sections 24 can be moved respectively away or toward one another. The instrument 20 operates similar to forceps or scissors.

Each jaw section 24 comprises a tissue contact surface 27. The two tissue contact surfaces 27 are facing one another and are configured to hold or clamp biological tissue therebetween. In an open position I the two shank sections 25 and the two jaw sections 24 are pivoted away from one another around hinge 21 (FIG. 1). In a closed position II the shank sections 25 or jaw sections 24 and/or the tissue contact surfaces 27 are substantially orientated parallel to one another. The tissue contact surfaces 27 have minimum distance from one another in the closed position II. In the closed position II biological tissue can be clamped or held between the jaw sections 24 or tissue contact surfaces 27.

In addition, the surgical instrument comprises a tool 30 that is movably or slidably supported in a longitudinal direction L in a tool channel 31 in or on the first jaw 22. The tool 30 is particularly a knife 32 that can be moved between an extended position (FIG. 3) and a retracted position (FIGS. 1 and 2). In the extended position tissue clamped between the tissue contact surfaces 27 or between the jaw sections 24 can be cut or separated.

For movement of tool 30 and according to the example, the knife 32 in longitudinal direction L, the instrument 20 comprises an actuation device 33. The actuation device 33 has an actuation element 34 that is pivotably supported around the pivot axis S on the first jaw 22. The pivot axis S extends orthogonal to the longitudinal direction substantially parallel to a hinge axis defined by hinge 21. The actuation element 34 can be arranged next to the two jaws 22, 23 with view parallel to the pivot axis S. Originating from the pivot axis S the actuation element 34 extends to a free end that can be actuated by a user with the hand or a finger in order to pivot the actuation element 34 around the pivot axis S.

The actuation device 33 has in addition a drive element 35 that is pivotably supported around the pivot axis S. In FIGS. 1 to 3 the drive element 35 is only schematically illustrated by an arm or a lever.

The drive element 35 is movably coupled with tool 30 and, according to the example, knife 32 by means of a transmission device 36. The transmission device 36 comprises at least one transmission element 37 and is, according to the example, formed by one single transmission element 37 that is configured as connecting rod and connects or couples tool 30 with drive element 35. Via transmission element 37 the pivot movement of the drive element 35 around the pivot axis S is converted in a linear movement of tool 30 in longitudinal direction L. For this transmission element 37 can be connected in a hinged manner or rotatably with drive element 35 and/or tool 30 in an embodiment.

A coupling device 40 is also shown in FIGS. 1 to 3 in a highly schematic manner. The coupling device 40 can be in a decoupling condition (FIG. 1) and a coupling condition (FIGS. 2 and 3). In the decoupling condition the actuation element 34 can be pivoted around the pivot axis S without thereby influencing the pivot position of drive element 35 around pivot axis S. In the coupling condition actuation element 34 and drive element 35 are connected in a torque-proof manner with each other, such that a pivot movement of the actuation element 34 around the pivot axis S results in a pivot movement of drive element 35 around pivot axis S.

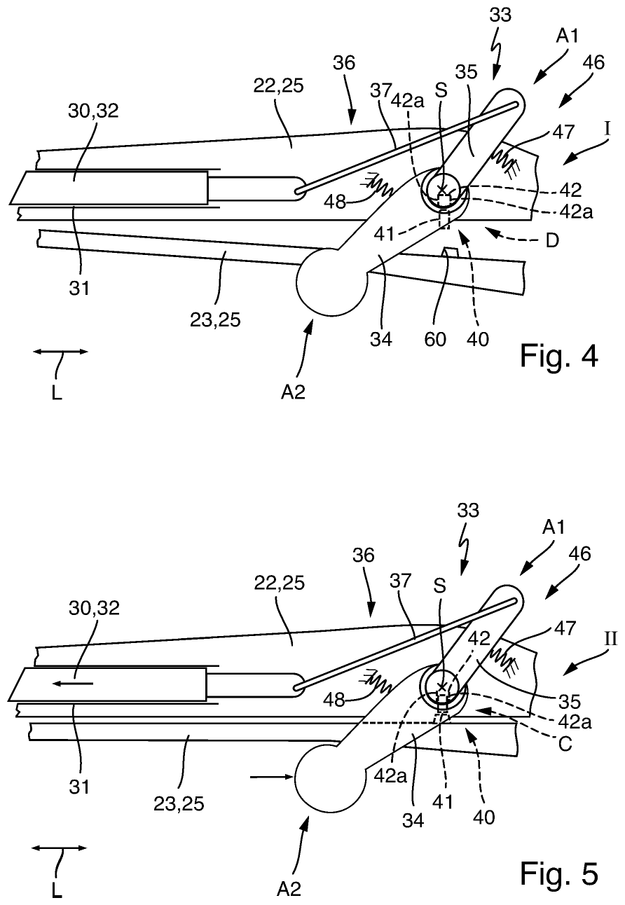

An embodiment of the actuation device 33 is schematically illustrated in FIGS. 4 and 5 based on a basic illustration. The coupling device 40 comprises, according to the example, a movable coupling element 41 that can be moved between a decoupling position D (FIG. 4) and a coupling position C (FIG. 5). In the coupling position C the coupling device 40 is in the coupling condition and in the decoupling position D the coupling device 40 is in the decoupling condition.

As schematically illustrated in FIGS. 4 and 5, drive element 35 comprises a coupling recess 42 that is orientated orthogonal or radial to the pivot axis S according to the example. The coupling element 41 is arranged on the actuation element 34. The coupling recess 42 is open on the side facing the actuation element 34. In the decoupling position D the coupling element 41 is located completely outside the coupling recess 42. In the coupling position C the coupling element 41 engages into coupling recess 42 and thereby establishes a rotational coupling or torque-proof connection between actuation element 34 and drive element 35. The coupling recess 42 is preferably limited by two flanks 42a that are arranged with distance opposite to each other in circumferential direction around pivot axis S.

The coupling element 41 is preferably linearly movably supported orthogonal or radial to the pivot axis S and/or pivotably supported. At least the end section of the movement path of the coupling element 41 adjoining the coupling position C can be orientated orthogonal to the pivot axis S.

In the decoupling position D of coupling element 41 the actuation element 34 and the drive element 35 can be moved independent from one another around the pivot axis S. This means that a pivot movement of the actuation element 34 around the pivot axis S does not change the pivot position of the drive element 35. If the coupling element 41, however, is in the coupling position C, a pivot movement of actuation element 34 results in a pivot movement of drive element 35 that is in turn transferred into a linear movement of tool 30 by means of transmission element 37. In doing so, a tool 30 can be extended and engage the area of the jaw sections 24. For example, knife 32 can cut or separate biological tissue held between the jaw sections 24.

As also schematically illustrated in FIGS. 4 and 5, instrument 20 comprises a resetting device 46. The resetting device 46 is configured to urge the actuation element 34 and the drive element 35 in a respective initial position A1 or A2. For this the resetting device 46 comprises, for example, a first resetting element 47 that is connected with drive element 35 as well as a second resetting element 48 that is connected with actuation element 34. The resetting elements 47, 48 are respectively configured to create a resetting force and can be configured as spring elastic resetting elements 47, 48. The resetting elements 47, 48 can be, for example, formed by leaf springs, helical springs, spiral springs or the like. The resetting elements 47, 48 are supported on the shank section 25 of first jaw 22 and thus create a resetting force between first jaw 22 on one hand and actuation element 34 or drive element 35 on the other hand.

In the embodiment the first resetting element 47 urges the drive element 35 in a first initial position A1 and the second resetting element 48 urges the actuation element 34 in a second initial position A2. If actuation element 34 and drive element 35 are in their respective initial positions A1, A2, the coupling element 41 on the actuation element 34 is aligned with the coupling recess 42 on the drive element 35. The establishment of the coupling position C is possible. If the actuation element 34 is pivoted out of the second initial position A2 while the coupling element 41 is in the decoupling position D, establishment of the rotational coupling is no longer possible. The actuation element 34 has to be first moved back into the second initial position A2.

In the preferred embodiments illustrated here the coupling element 41 is actuated or moved from the decoupling position D into the coupling position C by second jaw 23 and, according to the example, shank section 25 of second jaw 23, as schematically apparent from FIG. 5. In the closed position II second jaw 23 is in contact with coupling element 41 and moves the coupling element 41 into coupling recess 42 such that the coupling position C is reached. This displacement is possible, if actuation element 34 and drive element 35 are in their respective initial positions A1, A2.

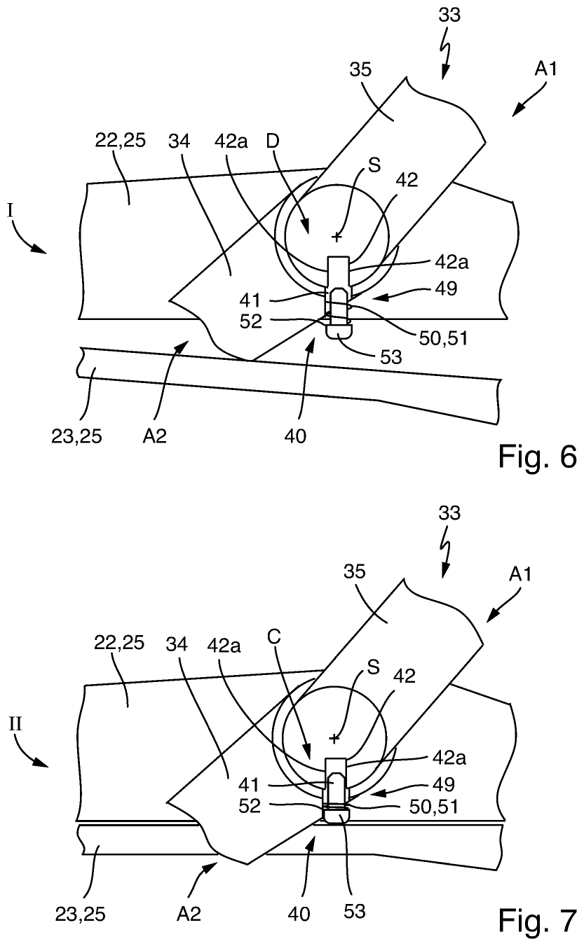

Different embodiments of the actuation device 33 are illustrated in FIGS. 6-14. In all embodiments a biasing device 49 is provided that is configured to urge coupling element 41 into the decoupling position D. For this biasing device 49 creates a biasing force on the coupling element 41 in direction toward the decoupling position D. The biasing force can be a tensile force and/or a pushing force. The biasing device 49 is arranged on actuation element 34 together with coupling element 41.

For creation of the biasing force the biasing device 49 preferably comprises a spring element 50. In an embodiment spring element 50 can be configured as helical spring (FIGS. 6 and 7). Thereby, for example, one end of the helical spring can be connected with coupling element 41 and the respective other end of helical spring 51 can be connected with actuation element 34. The connection can be configured as substance bond connection and/or adhesive connection. Between the two ends the helical spring 51 comprises a helically shaped extending spring section that can, for example, wind around a cylindrical coupling element 41.

The end connected with the coupling element 41 can abut against a ring step 52 of coupling element 41 or can be attached there. The ring step 52 can be formed by a transition from a cylindrical section assigned to the coupling recess 42 and a radially expanded head part 53 of coupling element 41. The head part 53 is facing the shank section 25 of second jaw 23.

In the open position of surgical instrument 20 (FIG. 6) the shank sections 25 of the two jaws 22, 23 are arranged with distance to one another. If jaws 22, 23 are moved into the closed position, the shank sections 25 approach each other and shank section 25 of second jaw 23 gets into contact with head part 53 of coupling element 41. During further movement in the direction toward the closed position II the coupling element 41 is moved against the biasing force of biasing device 49 and—according to the example helical spring 51—into the coupling recess 42 such that the coupling position C is established. Subsequently by pivot movement of the actuation element 34 a pivot movement of drive element 35 can be effected and in doing so, tool 30 or knife 32 can be extended or also retracted again.

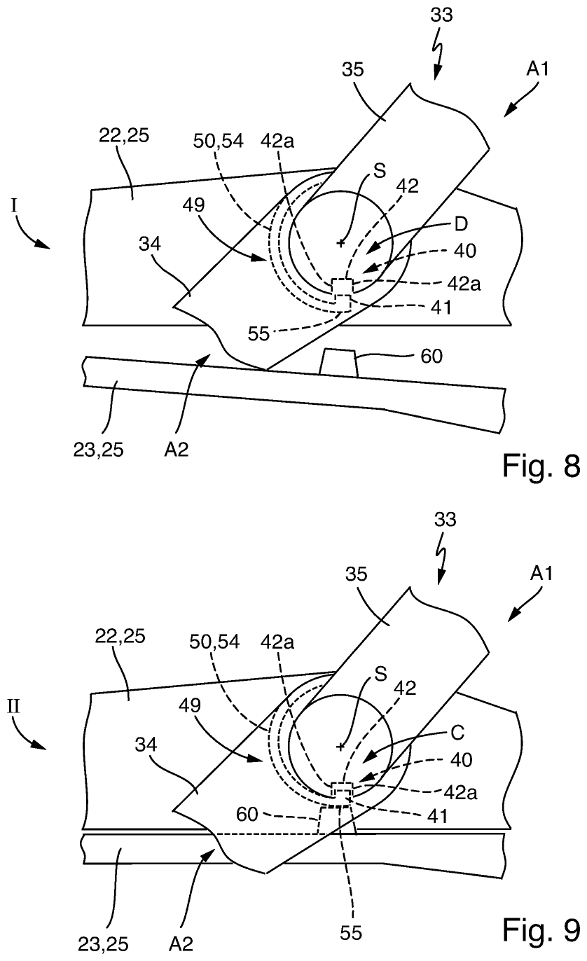

Another embodiment of the actuation device 33 is schematically illustrated in FIGS. 8 and 9. Basically reference can be made to the description of FIGS. 4-7. The substantial difference of the embodiment according to FIGS. 8 and 9 from the embodiments of the actuation device according to FIGS. 6 and 7 is that the spring element 50 of the biasing device 49 is formed as curved or arc-shaped flexible spring 54. The coupling element 41 is arranged on the flexible spring 54. In the embodiment the coupling element 41 is arranged on the free end 55 of flexible spring. The coupling element 41 can be non-releasably connected with flexible spring 54. Preferably flexible spring 54 and coupling element 41 are integrally formed.

Flexible spring 54 is attached with distance to the coupling element 41 and, according to the example, at its opposite attachment end to actuation element 34. Adjoining the attachment end the flexible spring 54 can be freely moved or elastically deformed. The free end 55 with the coupling element 41 is movable radial to the pivot axis S. In the decoupling position D coupling element 41 is arranged opposite coupling recess 42 orthogonal and preferably radial to the pivot axis S without engaging coupling recess 42. The flexible spring 54 extends arc-shaped around pivot axis S, e.g. in an angle area of at least 90° or at least 120° or at least 180°. The flexible spring 54 is arranged parallel to a plane that is orientated orthogonal to the pivot axis S.

Free end 55 of flexible spring 54 is facing second jaw 23 and accessible by second jaw 23. In order to be able to get into contact with second end 55 during closing movement in direction toward the closed position II, a projection 60 can be present on second jaw 23 that projects in direction toward first jaw 22 or pivot axis S away from second jaw 23. It is configured to actuate the flexible spring 54 in the area of the free end 55 and to establish the coupling position C in the closed position II of jaws 22, 23 (FIG. 9).

The particularity in this embodiment according to FIGS. 8 and 9 is that the biasing device 49 forms one unit with coupling element 41, preferably an integral unit. The operating principle corresponds otherwise to the embodiments described so far, such that reference can be made to the description above.

In FIG. 15 an embodiment of the actuation device 33 is illustrated that is modified compared with FIGS. 8 and 9. The coupling element 41 is analog to the embodiment of FIGS. 8 and 9 arranged on a flexible spring 54 that is attached on the drive element 35 in this embodiment. The drive element 35 comprises an opening or support 61 by means of which the coupling element 41 is slidably supported in direction toward the pivot axis S against the force of flexible spring 54. For example, support 61 can be a through-hole. The coupling element 41 can have the shape of a pin in this embodiment. In circumferential direction around the pivot axis S the coupling element 41 is supported via support 61 on drive element 35. The coupling recess 42 is provided on the actuation element 34.

In the open position I of jaws 22, 23, the coupling element 41 is in the decoupling position D. It is disengaged from the coupling recess 42 such that the actuation element 34 can be moved around the pivot axis S without initiating a movement of the drive element 35. The actuation element 34 can be urged by means of the resetting device 46—and according to the example the second resetting element 48—in the second initial position A2. In this embodiment the resetting device 46 does not comprise a resetting element in order to urge the drive element 35 into the first initial position A1. The drive element 35 is kept in the first initial position A1 and locked or secured against movement around pivot axis S, e.g. by means of coupling element 41. For this purpose coupling element 41 engages a locking opening 62 on the first jaw 22, e.g. with its end facing away from the coupling recess 42.

If jaws are moved from the open position I into the closed position II, coupling element 41 is disengaged by means of projection 60 on the second jaw 23 from locking opening 62 and engages coupling recess 42 such that coupling element 41 takes the coupling position C (not illustrated). As in the other embodiments, with coupling element 41 being in the coupling position C actuation element 34 and drive element 35 move together around pivot axis S, if the actuation element 34 is manually actuated. In this condition the resetting force of resetting device 46 does not only act on the actuation element 34, but also on the drive element 35.

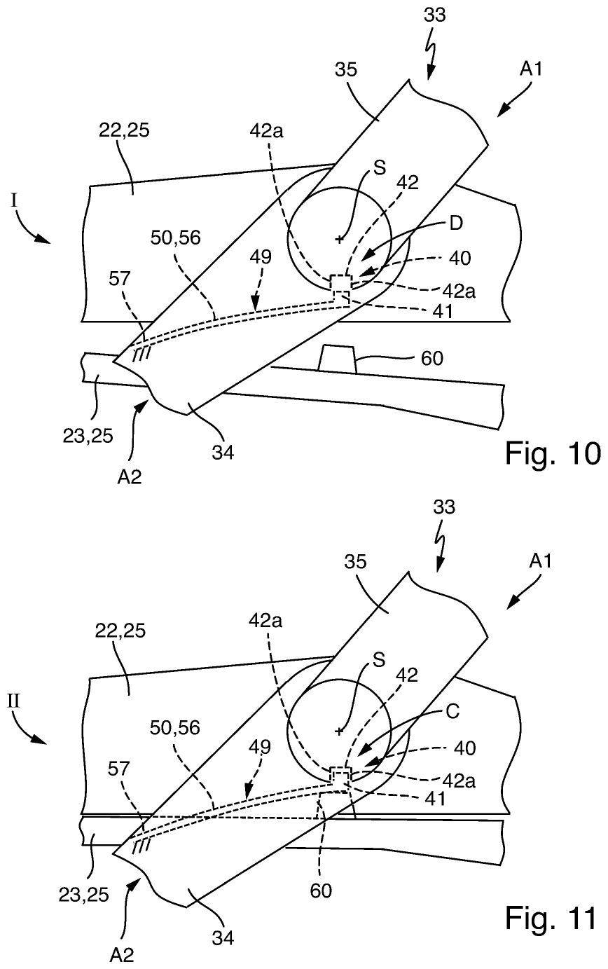

In FIGS. 10 and 11 another embodiment of the actuation device 33 is schematically illustrated. The essential difference compared with the embodiment according to FIGS. 8 and 9 is that the spring element 50 is configured as leaf spring 56 that extends in its unactuated initial condition in the decoupling position D straight or slightly curved from an attachment end 57 attached to the actuation element 34 up to a free end 55. On the free end 55 the coupling element 41 is arranged and can form a unit with leaf spring 56, as in the preceding embodiment. For forming the unit the coupling element 41 can be non-releasably connected with leaf spring 56 or can be integrally configured with leaf spring 56.

The operating principle for establishing the coupling condition or coupling position C corresponds to the preceding embodiments so that reference can be made to the above description.

The distance of leaf spring 56 from pivot axis S is preferably continuously increasing originating from free end 55 along the extension of leaf spring 56 to the attachment end 57. A straight line that connects attachment end 57 with free end 55 extends offset from the pivot axis and does not extend, according to the example, through drive element 35.

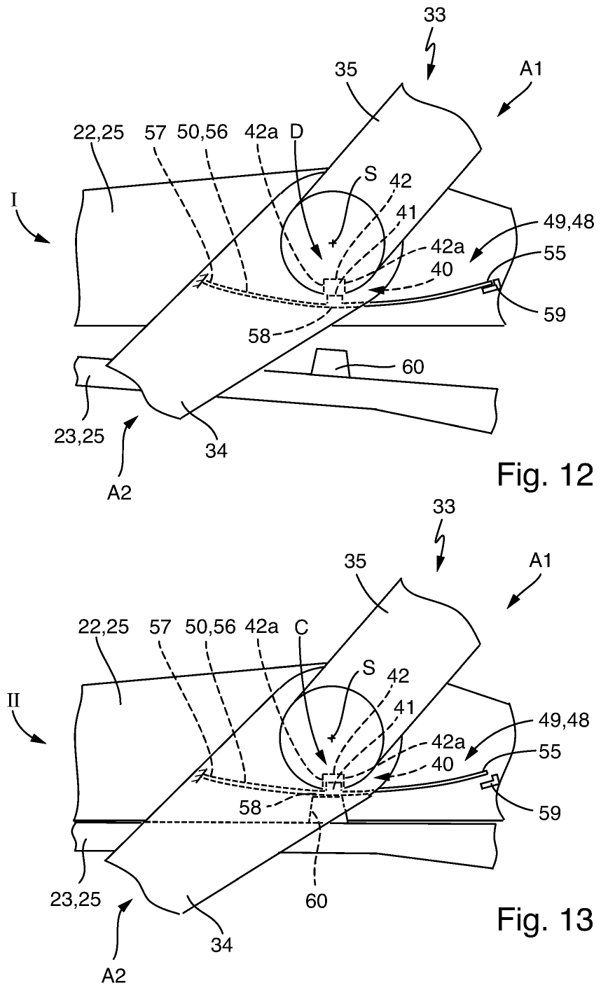

In the embodiment of actuation device 33 illustrated in FIGS. 12 to 14 spring element 50 is again formed by a leaf spring 56 similar to the embodiment according to FIGS. 10 and 11. The essential difference to the preceding embodiment is that leaf spring 56 extends in an arc-shaped curved manner from the attachment end 57 attached with the actuation element 34 up to the free end 55, wherein the coupling element 41 is arranged in a central section 58 of leaf spring 56 between the two ends 55, 57.

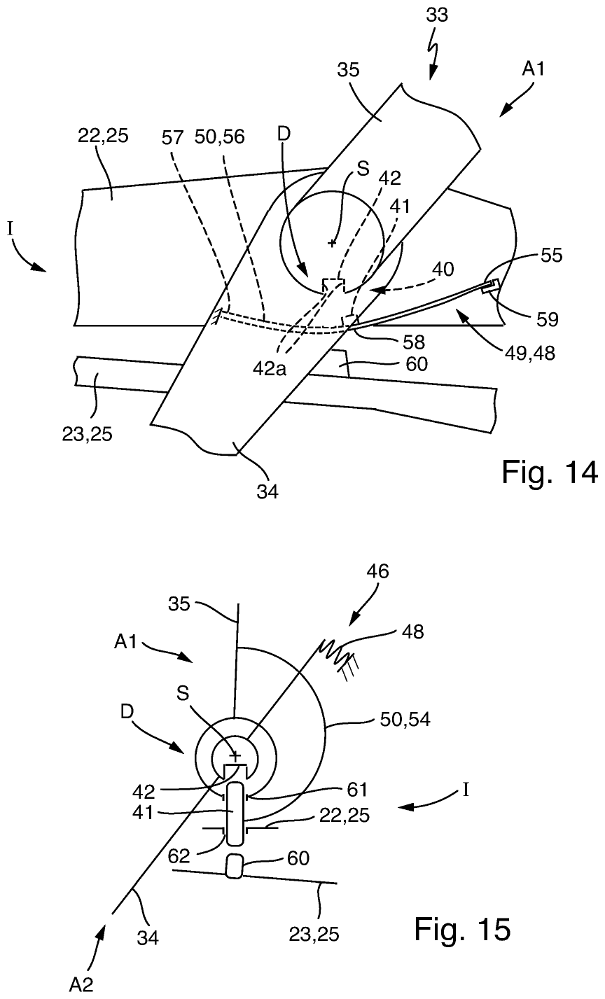

Free end 55 is supported in the decoupling position D on a counter support 59 that is arranged on the shank section 25 of first jaw 22. In the decoupling position D coupling element 41 is located completely outside of coupling recess 42 and is arranged opposite thereto in a direction orthogonal or radial to pivot axis S. The curvature of leaf spring 56 is concave on the side facing pivot axis S and accordingly convex on the side facing away from pivot axis S. In the decoupling position D leaf spring 56 of biasing device 49 concurrently forms the second resetting element 48 to urge the actuation element 34 back into the second initial position A2. This effect is schematically illustrated in FIG. 14. If in the decoupling position D actuation element 34 is pivoted away from second initial position A2, leaf spring 56 is supported between attachment end 57 and counter support 59 and is thus elastically deformed compared with its initial condition (FIG. 12) and is bent according to the example. In doing so, a resetting effect or resetting torque around pivot axis S is created that acts on actuation element 34 and urges it back in direction toward the second initial position A2. As apparent from FIG. 14, no coupling condition or coupling position C can be established while actuation element 34 is displaced from second initial position A2.

If actuation element 34 is in the second initial position A2 and drive element 35 is in the first initial position A1, establishment of the coupling condition or coupling position C is allowed. If the jaws 22, 23 are moved from the open position I into the closed position II, shank section 25 of second jaw 23—and projection 60 according to the example—gets in contact with central section 58 of leaf spring 56 and moves coupling element 41 into coupling recess 42. The coupling position C is established. As apparent from FIG. 13, thereby the free end 55 of leaf spring 56 is disengaged concurrently from counter support 59. Free end 55 is lifted due to the contact with second jaw 23 from counter support 59 when the closed position II is reached. In doing so, it is achieved that the common movement of actuation element 34 and the coupled drive element 35 around pivot axis S can be carried out in a manner unimpeded by counter support 59.

If the coupling condition or a coupling position C is established and if subsequently a movement of the actuation element 34 and the drive element 35 around pivot axis S is carried out, coupling element 41 seated on actuation element 34 is subject to a force in circumferential direction around the pivot axis S inside the coupling recess 42 on drive element 35, such that the coupling position C is maintained. Only if drive element 35 reaches the first initial position A1 and the actuation element 34 reaches the second initial position A2, the coupling position C can be suspended, if the jaws 22, 23 take the open position I or are brought into the open position I. Outside of the initial positions A1, A2 the force acting on coupling element 41 by biasing device 49 is sufficiently low, so that due to the clamping force in circumferential direction around the pivot axis S outside of the initial positions A1, A2, no suspension of the coupling position C occurs.

The resetting device 46 mentioned in connection with FIGS. 4 and 5 can be present in all embodiments, particularly the embodiments according to FIGS. 8-11. In the embodiments according to FIGS. 12-14 the second resetting element 48 is concurrently formed by the biasing device 49 and, according to the example, the leaf spring 56. In addition, the first resetting element 47 can be provided according to FIGS. 4, 5.

In all embodiments a projection 60 or another suitable geometry can be formed on the shank section 25 of second jaw 23, such that in the closed position II movement of coupling element 41 into the coupling recess 42 is allowed for creation of the coupling position C.

In all embodiments the surgical instrument 20 can be configured as electrosurgical instrument as an option. It can be, for example, a monopolar or bipolar electrosurgical instrument. For this purpose instrument 20 can be connected with an electrical line by means of an electrical connection device 65 (FIG. 1-3). Inside of one of the jaws and, according to the example the second jaw 23, an electrical conductor device extends by means of which tool 30 and/or at least one of the tissue contact surfaces 27 can be electrically connected. Clamped tissue can be electrosurgically treated in this manner, e.g. for the purpose of vessel sealing.

Embodiments of the invention refer to a surgical instrument 20 having a first jaw 22 and a second jaw 23 that are arranged on one another in a hinged manner. A tool 30 is slidably supported in a longitudinal direction L in a tool channel 31, preferably in or on first jaw 22. By means of an actuation device 33, tool 30 can be moved in longitudinal direction L. An actuation element 34 and a drive element 35 are part of the actuation device 33 and are pivotably supported around pivot axis S, preferably on the first jaw 22. By means of a coupling device 40, a rotational coupling can be established between actuation element 34 and drive element 35. The torque-proof coupling is preferably established in that the jaws 22, 23 are moved in the closed position II.

1. A surgical instrument comprising:
a first jaw and a second jaw that are supported on one another in a hinged manner and that can be pivoted between an open position and a closed position relative to each other;
a tool that is movably supported in a longitudinal direction in a guided manner on or in the first jaw;
an actuation element and a drive element that are pivotably supported around a pivot axis on first jaw, whereby the drive element is movably coupled with the tool; and
a coupling device comprising a coupling element configured to be moved between a coupling position and a decoupling position and configured to establish a rotational coupling between the actuation element and the drive element in the coupling position and to allow a relative rotation between the actuation element and the drive element in the decoupling position.

2. The surgical instrument according to claim 1, wherein the coupling element is movable by means of the second jaw.

3. The surgical instrument according to claim 1, wherein the second jaw is configured to move the coupling element out of the decoupling position and into the coupling position if the first jaw and the second jaw are pivoted relative to one another into the closed position.

4. The surgical instrument according to claim 1, wherein the drive element comprises a coupling recess into which the coupling element projects in coupling position.

5. The surgical instrument according to claim 1, wherein the coupling element is translationally movable between the coupling position and the decoupling position.

6. The surgical instrument according to claim 1, wherein the coupling element is configured to be pivoted between the coupling position and the decoupling position.

7. The surgical instrument according to claim 1, further comprising a biasing device configured to create a biasing force on the coupling element to cause the coupling element to move into the decoupling position.

8. The surgical instrument according to claim 7, wherein the biasing device comprises a spring element for creating the biasing force.

9. The surgical instrument according to claim 8, wherein the spring element and the coupling element are immovably coupled together relative to each other.

10. The surgical instrument according to claim 9, wherein the spring element and the coupling element are integrally formed.

11. The surgical instrument according to claim 8, wherein the spring element comprises one selected from the group of a flexible spring that extends in an arc-shaped manner around the pivot axis and a leaf spring.

12. The surgical instrument according to claim 8, wherein the spring element comprises a helical spring.

13. The surgical instrument according to claim 1, further comprising a resetting device configured to urge the actuation element and/or the drive element into an initial position.

14. The surgical instrument according to claim 13, wherein the resetting device comprises:
a first resetting element for urging the drive element into the initial position; and
a second resetting element for urging the actuation element into the initial position.

15. The surgical instrument according to claim 14, wherein first resetting element comprises an elastically deformable transmission element arranged between the drive element and the tool.
